(19) United States
(12) Patent Application Publication
(10) Pub. No.: US 2025/0207951 A1
(43) Pub. Date: Jun. 26, 2025

(54) ROTATION DETECTION METHOD AND APPARATUS, AND MEDICAL DEVICE

(71) Applicant: GE Precision Healthcare LLC, Waukesha, WI (US)

(72) Inventors: Ruiyu Wang, Beijing (CN); Lina Liu, Beijing (CN); Zhiqiang Liu, Beijing (CN)

(21) Appl. No.: 18/986,512

(22) Filed: Dec. 18, 2024

(30) Foreign Application Priority Data
Dec. 21, 2023 (CN) 202311774100.5

(51) Int. Cl. G01D 5/244; G01D 5/245; G01P 3/481

(52) U.S. Cl. CPC G01D 5/24466; G01D 5/245; G01P 3/481

(57) ABSTRACT

Embodiments of the present application provide a rotation detection method and apparatus and a medical device for performing rotation detection on a scanning gantry driven by a motor. The rotation detection method includes detecting at least one rotation of the scanning gantry by using a detector provided on the scanning gantry, and generating a pulse signal, counting rotations of the motor by using an encoder provided on the motor, and determining whether a rotation fault has occurred according to the number of counts within a time between two consecutive pulse signals. According to the embodiments of the present application, it is possible to detect whether loss of counts or a slippage fault has occurred. A closed detection loop can be formed even if an encoder on the scanning gantry is omitted, thereby reducing costs while ensuring detection accuracy.

### CROSS-REFERENCE TO RELATED APPLICATIONS

This application claims priority to Chinese Application No. 202311774100.5, filed on Dec. 21, 2023, the disclosure of which is incorporated herein by reference in its entirety.

### TECHNICAL FIELD

The present application relates to the technical field of medical devices, and relates in particular to a rotation detection method and apparatus, and a medical device.

### BACKGROUND

In a computed tomography (CT) process, an X-ray emitter and a detector in a scanning gantry rotate at a high speed about an axis of the scanning gantry, and meanwhile, a scanning table drives an object to be scanned (also referred to as a scan subject) through an X-ray plane or a ray beam to scan the object. After data of X-rays passing through the object is acquired by using the detector, the acquired X-ray data is processed to acquire projection data. The projection data may be used to reconstruct a CT image. Complete projection data can be used to reconstruct an accurate CT image for medical diagnosis.

At present, scanning gantries of CT devices are mostly driven by motors.

It should be noted that the above introduction of the background is only for the convenience of clearly and completely describing the technical solutions of the present application, and for the convenience of understanding for those skilled in the art.

### SUMMARY

The inventors have found that, in existing CT devices, the problem of rotation fault of the scanning gantry causes a CT image to rotate, thereby affecting medical diagnosis. For example, for the scanning gantry driven by the motor, when loss of motor rotation counts occurs, a rotation fault will occur in the scanning gantry, thereby affecting normal operation of the CT device.

In view of at least one of the above technical problems, embodiments of the present application provide a rotation detection method and apparatus, and a medical device. The rotation detection method can effectively detect a rotation fault of a scanning gantry, thereby ensuring that a medical device operates normally and reducing use costs. The rotation detection method and apparatus of the present application can be used in medical imaging devices (e.g., a CT device), and can also be used in other medical devices comprising scanning gantries driven by motors.

According to an aspect of embodiments of the present application, provided is a rotation detection method, for performing rotation detection on a scanning gantry driven by a motor, the method including detecting at least one rotation of the scanning gantry by using a detector provided on the scanning gantry, and generating a pulse signal, counting rotations of the motor by using an encoder provided on the motor, and determining whether a rotation fault has occurred according to the number of counts within a time between two consecutive pulse signals.

Therefore, upon at least one complete rotation, the detector on the scanning gantry generates a pulse. It can be determined, according to the number of counts of rotations of the motor between two consecutive pulses, whether loss of counts or a slippage fault has occurred. A closed detection loop can be formed even if an encoder on the scanning gantry is omitted, thereby reducing costs while ensuring detection accuracy.

In some embodiments, the detector generates the pulse signal of one period each time the scanning gantry performs one complete rotation. In some embodiments, the rotation detection method further includes, when it is determined that a rotation fault has occurred in the scanning gantry, stopping rotation of the scanning gantry and aborting a scan. Thus, restarting the scan can reduce the probability of a scanning gantry fault to a certain degree and avoid an excessive radiation dose to a subject under examination. In some embodiments, the determining whether a rotation fault has occurred includes comparing the number of counts within the time between the two consecutive pulse signals with a first threshold, and when a comparison result is not within a first predetermined range, determining that a rotation fault has occurred. Therefore, a rotation fault is determined by comparing the number of motor rotation counts with the set threshold, so that a fault can be detected via a simple solution.

In some embodiments, the rotation detection method further includes determining a rotation mode of the scanning gantry, and adjusting, according to the determined rotation mode of the scanning gantry, the first threshold corresponding to the comparison.

In some embodiments, rotation modes of the scanning gantry include a constant speed mode, an acceleration mode, and a deceleration mode. The constant speed mode corresponds to a preset threshold V11, the acceleration mode corresponds to a preset threshold V12, the deceleration mode corresponds to a preset threshold V13, V12=K11*V11, and V13=K12*V11, wherein K11 and K12 are adjustment coefficients. In some examples, the rotation modes of the scanning gantry include a first constant speed mode, a second constant speed mode, and a third constant speed mode. The first constant speed mode corresponds to a preset threshold V21, the second constant speed mode corresponds to a preset threshold V22, the third constant speed mode corresponds to a preset threshold V3, V2=K21*V21, and V3=K22*V21, wherein K21 and K22 are adjustment coefficients. Thus, the threshold is adjusted for different modes, so that it is possible to adapt to different scanning situations of the scanning gantry, thereby adaptively performing the rotation detection.

In some embodiments, the rotation detection method further includes totaling a plurality of comparison results/. and when a totaling result is not within a second predetermined range, determining that a rotation fault has occurred. Therefore, the determination is performed according to the totaling, so that the issue of an accumulated error can be further resolved, thereby improving the accuracy of the determination of the rotation fault. In some embodiments, when a rotation mode of the scanning gantry is a constant speed mode, the totaling is enabled, and when the rotation mode of the scanning gantry is not the constant speed mode, the totaling is disabled. Therefore, the totaling is enabled or disabled depending on the mode, so that the accuracy of the determination of the accumulated error can be further improved, thereby improving the accuracy of the determination of the rotation fault.

According to another aspect of the embodiments of the present application, provided is a rotation detection apparatus, for performing rotation detection on a scanning gantry driven by a motor, the apparatus including a pulse generation unit, for detecting at least one rotation of the scanning gantry by using a detector provided on the scanning gantry, and generating a pulse signal. A motor counting unit, for counting rotations of the motor by using an encoder provided on the motor, and a fault determination unit, for determining whether a rotation fault has occurred according to the number of counts within a time between two consecutive pulse signals.

According to another aspect of the embodiments of the present application, provided is a medical device, provided with a scanning gantry driven by a motor, the medical device being provided with the rotation detection apparatus according to the embodiments of the foregoing aspect.

One of the beneficial effects of the embodiments of the present application is, upon at least one complete rotation, the detector on the scanning gantry generates a pulse. It can be determined, according to the number of counts of rotations of the motor between two consecutive pulses, whether loss of counts or a slippage fault has occurred. A closed detection loop can be formed even if an encoder on the scanning gantry is omitted, thereby reducing costs while ensuring detection accuracy.

With reference to the following description and drawings, specific implementations of the present application are disclosed in detail, and the means by which the principles of the present application can be employed are illustrated. It should be understood that the embodiments of the present application are not limited in scope thereby. Within the scope of the spirit and clauses of the appended claims, the embodiments of the present application include many changes, modifications, and equivalents.

The features described and/or illustrated for one implementation may be used in one or more other implementations in the same or similar manner, be combined with features in other embodiments, or replace features in other implementations.

It should be emphasized that the terms “include/comprise” when used herein refer to the presence of features, integrated components, steps, or assemblies, but do not preclude the presence or addition of one or more other features, integrated components, steps, or assemblies.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other drawings according to the drawings without the exercise of inventive effort. In the drawings:

FIG. 1 is a schematic diagram of a rotation detection method according to an embodiment of the present application;

FIG. 2 is a schematic diagram of a detector of a scanning gantry according to an embodiment of the present application;

FIG. 3 is a schematic diagram of rotation detection according to an embodiment of the present application;

FIG. 4 is a schematic diagram of a motor rotation count according to an embodiment of the present application;

FIG. 5 a schematic diagram of counting of constant speed rotations of a motor at different speeds according to an embodiment of the present application;

FIG. 6 is a schematic diagram of a rotation detection apparatus according to an embodiment of the present application;

FIG. 7 is a schematic diagram of a medical image processing device according to an embodiment of the present application; and

FIG. 8 is a schematic diagram of a medical device according to an embodiment of the present application.

### DETAILED DESCRIPTION

The foregoing and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of”' or “a class of” rather than being limited to the meaning of “one”. Furthermore, the term “the” should be construed as including both the singular and plural forms, unless otherwise specified in the context. In addition, the term “according to” should be construed as “at least in part according to . . .” and the term “on the basis of” should be construed as “at least in part on the basis of . . .”, unless otherwise specified in the context.

The features described and/or illustrated for one implementation may be used in one or more other implementations in the same or similar manner, be combined with features in other embodiments, or replace features in other implementations. The term “include/comprise” when used herein refers to the presence of features, integrated components, steps, or assemblies, but does not preclude the presence or addition of one or more other features, integrated components, steps, or assemblies.

The rotation detection method and apparatus described herein are applicable to various medical devices including, but not limited to, a computed tomography (CT) device, a positron emission tomography-computed tomography (PET-CT) device, or any other suitable medical imaging device. For example, a CT device uses X-rays to perform continuous cross-sectional scanning around a certain part of a scan subject, and the X-rays that pass through a section are received by a detector and transformed into visible light, or a received photon signal is directly converted to perform image reconstruction after a series of processing. The rotation detection method and apparatus of the embodiments of the present application are applicable to all of these medical devices.

The embodiments of the present application are specifically described below.

An embodiment of the present application provides a rotation detection method, for performing rotation detection on a scanning gantry driven by a motor.

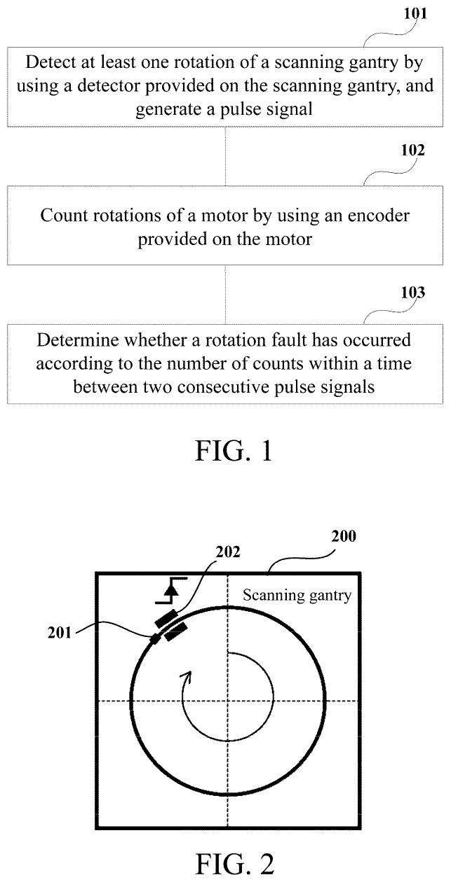

FIG. 1 is a schematic diagram of a rotation detection method according to an embodiment of the present application. As shown in FIG. 1, the method includes at step 101, detecting at least one rotation of the scanning gantry by using a detector provided on a scanning gantry, and generating a pulse signal, at step 102, counting rotations of the motor by using an encoder provided on the motor, and at step 103, determining whether a rotation fault has occurred according to the number of counts within a time between two consecutive pulse signals.

It should be noted that FIG. 1 merely schematically illustrates the embodiment of the present application, but the present application is not limited thereto. For example, some of the above steps may be performed simultaneously, or may be performed in a sequential order. The order of execution between operations may be appropriately adjusted. In addition, some other operations may be added or some operations may be omitted. Those skilled in the art may make appropriate variations according to the above content, rather than being limited to the above disclosure of FIG. 1.

In the embodiments of the present application, upon at least one complete rotation, the detector on the scanning gantry generates a pulse. It can be determined, according to the number of counts of rotations of the motor between two consecutive pulses, whether loss of counts or a slippage fault has occurred. A closed detection loop can be formed even if an encoder on the scanning gantry is omitted, thereby reducing costs while ensuring detection accuracy.

In some embodiments, the detector generates the pulse signal of one period, i.e., a home flag signal of the scanning gantry, each time the scanning gantry performs one complete rotation. The present application is not limited thereto, and other detection components may also be used.

FIG. 2 is a schematic diagram of a detector of a scanning gantry according to an embodiment of the present application. As shown in FIG. 2, the detector is, for example, a return-to-zero detection apparatus provided on a scanning gantry 200. The detector includes a home flag 201 mounted on a rotating portion of the scanning gantry 200 and a photoelectric sensor 202 mounted on a fixed portion of the scanning gantry 200. Each time the rotating portion of the scanning gantry 200 performs one complete rotation, the home flag 201 passes through the photoelectric sensor 202 once, and correspondingly, the return-to-zero detection apparatus generates a pulse signal of one period.

In some embodiments, the rotations of the motor are counted by using the encoder provided on the motor. Regarding how the encoder performs rotation detection, reference may be made to the related art, and details will not be described herein again.

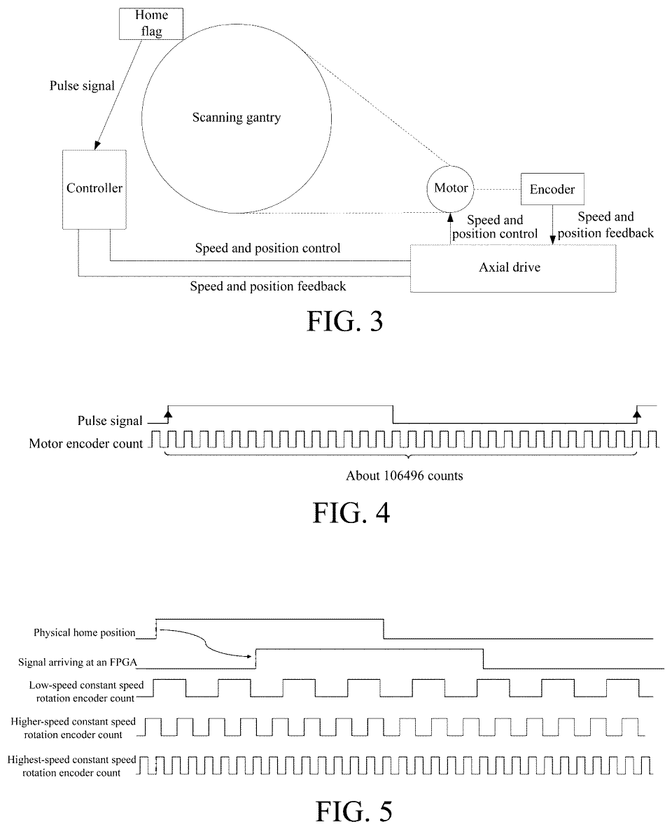

FIG. 3 is a schematic diagram of rotation detection according to an embodiment of the present application. As shown in FIG. 3, at least one rotation of the scanning gantry may be detected by using the home flag provided on the scanning gantry, and a pulse signal may be generated. Rotations of the motor by be counted using the encoder provided on the motor. A controller determines whether a rotation fault has occurred according to the number of counts within a time between two consecutive pulse signals.

As shown in FIG. 3, the controller may receive information (the pulse signal) from the scanning gantry, and receive information (e.g., speed and position information measured by the encoder) from the motor. Therefore, a closed detection loop can be formed even if an encoder on the scanning gantry is omitted, thereby reducing costs while ensuring detection accuracy.

The embodiments of the present application have been schematically described above, and how to perform counting and how to determine a rotation fault will be further described below.

FIG. 4 is a schematic diagram of a motor rotation count according to an embodiment of the present application, and exemplarily shows some cases of counting according to the embodiment of the present application. As shown in FIG. 4, the encoder of the motor counts the rotations of the motor. The count may be based on, for example, a count of a gear mark of the motor, or one count is performed for each rotation of a photoelectric sensor or a magnetoelectric sensor in a coil of the motor. The time between two consecutive home pulse signals may correspond to the number of counts of rotations of the motor counted by the encoder of the motor. As shown in FIG. 4, for example, within one pulse signal period, a motor rotation count may be 106496.

In some embodiments, determining whether a rotation fault has occurred includes comparing the number of counts within the time between the two consecutive pulse signals with a first threshold, and when a comparison result is not within a first predetermined range, determining that a rotation fault has occurred.

For example, using FIG. 4 as an example, the first threshold may be set to 106500. In some scenarios, if the motor rotation count within one pulse signal period is greater than 106500, it is determined that a rotation fault has occurred. If the motor rotation count within one pulse signal period is less than or equal to 106500, it is determined that no rotation fault has occurred.

As another example, using FIG. 4 as an example, the first threshold may be set to 106500. In some other scenarios, if the motor rotation count within one pulse signal period is less than 106500, it is determined that a rotation fault has occurred. If the motor rotation count within one pulse signal period is greater than or equal to 106500, it is determined that no rotation fault has occurred.

As another example, using FIG. 4 as an example, the first threshold may be set to [106500, 106600], and if the motor rotation count within one pulse signal period is within the range of 106500 to 106600, it is determined that no rotation fault has occurred. If the motor rotation count within one pulse signal period is not within the range of 106500 to 106600, it is determined that a rotation fault has occurred.

Therefore, a rotation fault is determined by comparing the number of motor rotation counts with the set threshold, so that a fault can be detected via a simple solution.

In some embodiments, determination may be performed on rotations by using a plurality of thresholds for different cases. For example, the rotation detection method further includes determining a rotation mode of the scanning gantry, and adjusting, according to the determined rotation mode of the scanning gantry, the first threshold corresponding to the comparison.

For example, rotation modes of the scanning gantry include a constant speed mode, an acceleration mode, and a deceleration mode. The constant speed mode corresponds to a preset threshold V11. The acceleration mode corresponds to a preset threshold V12. The deceleration mode corresponds to a preset threshold V13. V12=K11*V11. V13=K12*V11. K11 and K12 are adjustment coefficients. Specific values of K11 and K12 are not limited in the present application, and may be determined according to, for example, empirical values or statistical values.

As another example, the rotation modes of the scanning gantry include a first constant speed mode, a second constant speed mode, and a third constant speed mode. The first constant speed mode corresponds to a preset threshold V21. The second constant speed mode corresponds to a preset threshold V22. The third constant speed mode corresponds to a preset threshold V23. V22=K21*V21. V23=K22*V21.

FIG. 5 a schematic diagram of counting of constant speed rotations of a motor at different speeds according to an embodiment of the present application. As shown in FIG. 5, for example, there is a delay between a physical home position of a pulse signal generated by the home flag and a position where the signal arrives at an FPGA (controller). The path delay of the pulse signal is almost fixed, and the time of the delay is less than a millisecond. As the rotational speed of the motor increases in the constant speed mode, the number of motor rotation counts per unit time increases. Thus, the thresholds for different rotation modes need to be adapted, e.g., multiplied by corresponding threshold coefficients.

For example, K21 and K22 are adjustment coefficients, where K21 is greater than 1, and K22 is less than 1, so that the preset threshold V22 corresponding to the highest constant speed mode (the second constant speed mode) is adjusted to K21*V21, and the preset threshold V23 corresponding to the low-speed constant speed mode (the third constant speed mode) is adjusted to K22*V21. In this way, the preset threshold V22 in the highest constant speed mode is greater than the preset threshold V21 in the higher constant speed mode (the first constant speed mode). The preset threshold V23 in the low-speed constant speed mode is less than the preset threshold V21 in the higher constant speed mode. Specific values of K11 and K12 are not limited in the present application, and may be determined according to, for example, empirical values or statistical values.

Furthermore, variations in the rotational speed of the scanning gantry 200 may result in variations in counts in different modes. However, due to great inertia of the scanning gantry 200, the variation in the rotational speed thereof is very small (far less than 1 r/s, where r represents each rotation, and s represents each second), so that in practical use, the threshold variation should not exceed, for example, 150 count specifications (approximately equivalent to 0.5 degrees of slippage of the scanning gantry) during different modes.

Thus, the threshold is adjusted for different modes, so that it is possible to adapt to different scanning situations of the scanning gantry, thereby adaptively performing the rotation detection. The above description is provided by using the highest constant speed mode, the higher constant speed mode, and the low-speed constant speed mode, but the present application is not limited thereto. For example, other modes and corresponding thresholds may also be set.

In some embodiments, the rotation detection method further includes totaling a plurality of comparison results, and when a totaling result is not within a second predetermined range, determining that a rotation fault has occurred. For example, comparison results within a long period of time may be statistically acquired, and accumulative detection may be performed on the basis of the results.

For example, the rotation detection may be performed in units of time period T, and accumulative determination may be performed in units of 10 T. For example, it is assumed that a count value in T0 is N0 and does not exceed the first threshold, therefore, is determined that no fault has occurred, . . ., it is assumed that a count value in T9 is N9 and does not exceed the first threshold, therefore, it is determined that no fault has occurred. However, the total value of N0 to N9 exceeds a second threshold, and in this case, it can be determined that a rotation fault has occurred. Therefore, the determination is performed according to the totaling, so that the issue of an accumulated error can be further resolved, thereby improving the accuracy of the determination of the rotation fault.

As another example, the rotation detection may be performed in units of time period T, and accumulative determination may be performed in units of 10 T. The second threshold for total fault number determination may be set to four. For example, it is assumed that the comparison result of T0 is that no fault has occurred, the comparison result of T1 is that a fault has occurred, . . . , and the comparison result of T9 is that no fault has occurred. Totally five faults are present in the ten comparison results corresponding to the time period of T0 to T9, which is greater than the second threshold, i.e., four, therefore, it can be determined that a rotation fault has occurred. It is therefore possible to exclude occasional cases according to the total number of faults, thereby further improving the detection accuracy.

As another example, the rotation detection may be performed in units of time period T, and accumulative determination may be performed in units of 10 T. A third threshold for determination of consecutive faults may be set to three. For example, it is assumed that the comparison result of T0 is that no fault has occurred, the comparison result of T1 is that a fault has occurred, . . ., and the comparison result of T9 is that no fault has occurred. If four consecutive faults are present in the ten comparison results corresponding to the time period of T0 to T9, which is greater than the third threshold, i.e., three, then it can be determined that a rotation fault has occurred. If totally four faults are present in the ten comparison results corresponding to the time period of T0 to T9, but there are only two consecutive faults, the number of consecutive faults is less than the third threshold, i.e., three, therefore, it can be determined that no rotation fault has occurred. It is therefore possible to exclude occasional cases according to the number of consecutive faults, thereby further improving the detection accuracy.

In some embodiments, when the rotation mode of the scanning gantry is the constant speed mode, the totaling is enabled; and when the rotation mode of the scanning gantry is not the constant speed mode, the totaling is disabled. For example, if the rotation mode of the scanning gantry is the constant speed mode, the accumulative determination may be enabled. If the rotation mode of the scanning gantry is not the constant speed mode, and is, for example, the acceleration mode or the deceleration mode, since the entire device is in a non-completely stable state, the accuracy of the determination may be affected, and the accumulative determination may be disabled. Therefore, the totaling is enabled or disabled depending on the mode, so that the accuracy of the determination of the accumulated error can be further improved, thereby improving the accuracy of the determination of the rotation fault.

In some embodiments, when it is determined that a rotation fault has occurred in the scanning gantry, rotation of the scanning gantry is stopped, and a scan is aborted. A scan performed by the scanning gantry is promptly aborted upon determination of a fault and is restarted, so that an operator can promptly and easily solve an issue, and a subject under examination is prevented from being unnecessarily affected by, for example, an excessive dose of CT radiation, etc. Thus, the probability of a scanning gantry fault can be reduced to a certain degree, and an excessive radiation dose to the subject under examination can be avoided.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

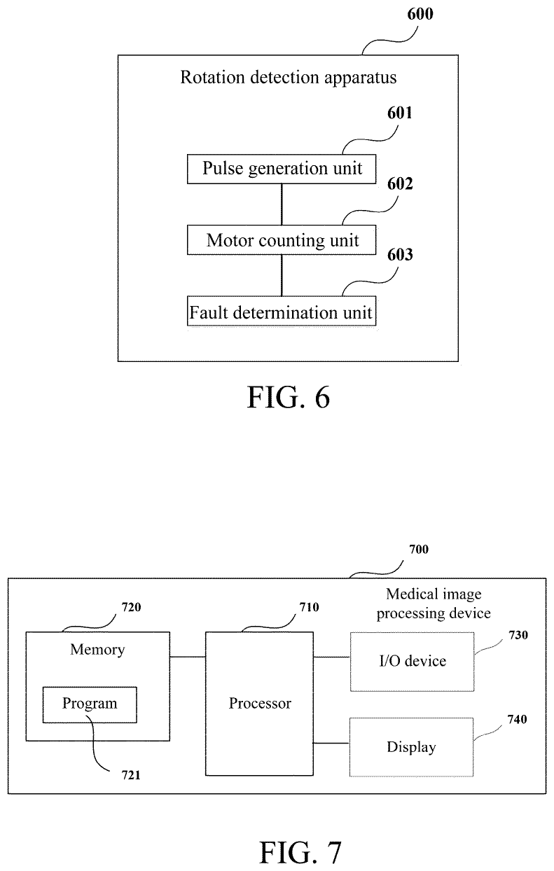

An embodiment of the present application further provides a rotation detection apparatus. FIG. 6 is a schematic diagram of a rotation detection apparatus according to an embodiment of the present application. As shown in FIG. 6, the rotation detection apparatus 600 includes a pulse generation unit 601, for detecting at least one rotation of a scanning gantry by using a detector provided on the scanning gantry, and generating a pulse signal, a motor counting unit 602, for counting rotations of the motor by using an encoder provided on the motor, and a fault determination unit 603, for determining whether a rotation fault has occurred according to the number of counts within a time between two consecutive pulse signals.

In some embodiments, for implementations of the pulse generation unit 601, the motor counting unit 602, and the fault determination unit 603, reference may be made to 101 to 103 of the aforementioned embodiments, which will not be described herein again.

An embodiment of the present application further provides a medical image processing device. FIG. 7 is a schematic diagram of a medical image processing device according to an embodiment of the present application. As shown in FIG. 7, the medical image processing device 1000 may include one or more processors (for example, central processing units (CPUs)) 710 and one or more memories 720. The memory 720 is coupled to the processor 710. The memory 720 may store the number of motor rotation counts and the like, and further input a control program 721 of the device, and the program 721 is executed under the control of the processor 710. The memory 720 may include, for example, a ROM, a floppy disk, a hard disk, an optical disk, a magneto-optical disk, a CD-ROM, or a non-volatile memory card.

In some embodiments, the functions of the rotation detection apparatus 600 are integrated into the processor 710 for implementation. The processor 710 is configured to implement the rotation detection method as described in the preceding embodiments. For an implementation of the processor 710, reference may be made to the foregoing embodiments. Details are not described herein again.

In some embodiments, the rotation detection apparatus 600 and the processor 710 are configured separately. For example, the rotation detection apparatus 600 can be configured to be a chip connected to the processor 710 and the functions of the rotation detection apparatus 600 can be achieved by means of the control of the processor 710.

In addition, as shown in FIG. 7, the medical image processing device 1000 may further include an input device 730, a display 740 (displaying a graphical user interface, and various data, image frames, or parameters generated during data acquisition and processing), etc. The functions of the foregoing components are similar to those in the prior art. Details are not described herein again. It should be noted that the medical image processing device 700 does not necessarily include all of the components shown in FIG. 7. In addition, the medical image processing device 700 may further include components not shown in FIG. 7, for which reference may be made to the related art.

The processor 710 may communicate with a medical device, the display, or the like in response to an operation of the input device, and may also control input actions and/or the state of the input device. The processor 710 may also be referred to as a microcontroller unit (MCU), microprocessor or microcontroller, or another processor apparatus and/or logic apparatus. The processor 710 may include a reset circuit, a clock circuit, a chip, a microcontroller, etc. The functions of the processor 710 may be integrated on a motherboard (e.g., the processor 710 is configured as a chip connected to a motherboard processor (CPU)) of the medical device, or may be configured independently of the motherboard, and the embodiments of the present application are not limited thereto.

An embodiment of the present application further provides a medical device. The medical device includes the medical image processing device 700 described in the foregoing embodiments, the contents of which are incorporated herein. In some embodiments, the medical device includes a computed tomography (CT) device and a PET-CT device, but the present application is not limited thereto, and the medical device may also be another device that may acquire medical imaging.

The functions of the processor of the medical image processing device 700 may be integrated into a motherboard (e.g., the processor is configured as a chip connected to a motherboard processor (CPU)) of the medical device, or may be configured independently of the motherboard, and the embodiments of the present application are not limited thereto.

In some embodiments, the medical device may further include other components. For details, reference may be made to the related art, which will not be described herein again. An exemplary description is given below by using a CT device as an example of the medical device.

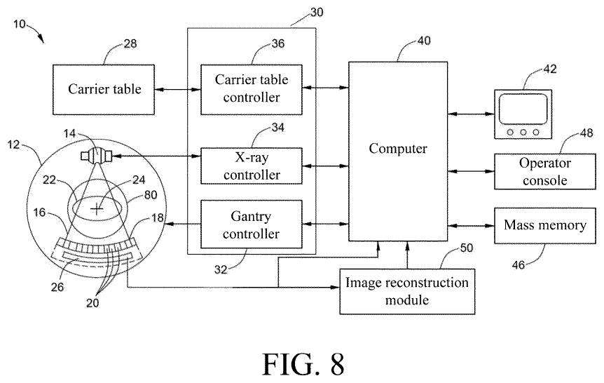

FIG. 8 is a schematic diagram of a medical device according to an embodiment of the present application. As shown in FIG. 8, the medical device is a CT system 10. The system 10 includes a gantry 12. An X-ray source 14 and a detector 18 are arranged opposite to each other on the gantry 12. The detector 18 is composed of a plurality of detector modules 20 and a data acquisition system (DAS) 26. The DAS 26 is configured to convert sampled analog data of analog attenuation data received by the plurality of detector modules 20 into digital signals for subsequent processing.

In some embodiments, the system 10 is used for acquiring, from different angles, projection data of a subject to be examined. Thus, components on the gantry 12 are used for rotating around a center of rotation 24 to acquire projection data. During rotation, the X-ray radiation source 14 is used to emit toward the detector 18 X-rays 16 that penetrate the subject to be examined. Attenuated X-ray beam data is preprocessed and then used as projection data of a target volume of the subject. An image of the subject to be examined may be reconstructed on the basis of the projection data. The reconstructed image may display internal features of the subject to be examined. These features include, for example, a lesion, the size, the shape, etc., of a body tissue structure. The center of rotation 24 of the gantry also defines the center of a scanning field 80.

The system 10 further includes an image reconstruction module 50. As described above, the DAS 26 samples and digitizes the projection data acquired by the plurality of detector modules 20. Next, the image reconstruction module 50 performs high-speed image reconstruction on the basis of the aforementioned sampled and digitized projection data. In some embodiments, the image reconstruction module 50 stores the reconstructed image in a storage device or a mass memory 46. Or, the image reconstruction module 50 transmits the reconstructed image to a computer 40 to generate information for diagnosing and evaluating patients.

Although the image reconstruction module 50 is illustrated as a separate entity in FIG. 8, in some embodiments, the image reconstruction module 50 may form a part of the computer 40. Or, the image reconstruction module 50 may not exist in the system 10, or the computer 40 may perform one or more functions of the image reconstruction module 50. Furthermore, the image reconstruction module 50 may be located at a local or remote location, and may be connected to the system 10 using a wired or wireless network. In some embodiments, computing resources having a centralized cloud network may be used for the image reconstruction module 50.

In some embodiments, the system 10 includes a control mechanism 30. The control mechanism 30 may include an X-ray controller 34 used to provide power and timing signals to the X-ray radiation source 14. The control mechanism 30 may further include a gantry controller 32 used to control the rotational speed and/or position of the gantry 12 on the basis of imaging requirements. The control mechanism 30 may further include a carrier table controller 36 configured to drive a carrier table 28 to move to a suitable position so as to position the subject to be examined in the gantry 12, so as to acquire the projection data of the target volume of the subject to be examined. Furthermore, the carrier table 28 includes a driving apparatus, and the carrier table controller 36 may control the carrier table 28 by controlling the driving apparatus.

In some embodiments, the system 10 further includes the computer 40, where data sampled and digitized by the DAS 26 and/or an image reconstructed by the image reconstruction module 50 is transmitted to a computer or the computer 40 for processing. In some embodiments, the computer 40 stores the data and/or image in a storage device such as a mass memory 46. The mass memory 46 may include a hard disk drive, a floppy disk drive, a CD-read/write (CD-R/W) drive, a digital versatile disc (DVD) drive, a flash drive, and/or a solid-state storage apparatus.

In some embodiments, the computer 40 transmits the reconstructed image and/or other information to a display 42, the display 42 being communicatively connected to the computer 40 and/or the image reconstruction module 50. In some embodiments, the computer 40 may be connected to a local or remote display, printer, workstation and/or similar device, for example, connected to such devices of medical institutions or hospitals, or connected to a remote device by means of one or a plurality of configured wires or a wireless network such as the Internet and/or a virtual private network. Furthermore, the computer 40 may provide commands and parameters to the DAS 26 and the control mechanism 30 (including the gantry controller 32, the X-ray controller 34, and the carrier table controller 36) on the basis of user provision and/or system definition, so as to control a system operation, such as data acquisition and/or processing.

In some embodiments, the computer 40 controls system operations on the basis of user input. For example, the computer 40 may receive user input such as commands, scanning protocols and/or scanning parameters, by means of an operator console 48 connected thereto. The operator console 48 may include a keyboard (not shown) and/or touch screen to allow a user to input/select commands, scanning protocols and/or scanning parameters.

In some embodiments, the system 10 may include or be connected to a picture archiving and communication system (PACS) (not shown in the figure). In some embodiments, the PACS is further connected to a remote system such as a radiology information system, a hospital information system 20, and/or an internal or external network (not shown) to allow operators at different locations to provide commands and parameters and/or access image data.

The method or process described in the aforementioned embodiments may be stored as executable instructions in a non-volatile memory in a computing device of the system 10. For example, the computer 40 may include executable instructions in the non-volatile memory and may apply the rotation detection method in the embodiments of the present application.

The computer 40 may be configured and/or arranged for use in different manners. For example, in some implementations, a single computer 40 may be used, and in other implementations, a plurality of computers 40 are configured to work together (for example, on the basis of distributed processing configuration) or separately, and each computer 40 is configured to process specific aspects and/or functions, and/or process data for generating models used only for a specific system 10. In some implementations, the computer 40 may be local (for example, in the same place as one or a plurality of systems 10, for example, in the same facility and/or the same local network). In other implementations, the computer 40 may be remote and thus only accessible by means of a remote connection (for example, by means of the Internet or other available remote access technologies). In a specific implementation, the computer 40 may be configured in a manner similar to that of cloud technology, and may be accessed and/or used in a manner substantially similar to that of accessing and using other cloud- based systems.

Once data (e.g., the number of motor rotation counts) is generated and/or configured, the data can be replicated and/or loaded into the medical system 10, which may be accomplished in different manners. For example, models may be loaded by means of a directional connection or link between the system 10 and the computer 40. In this regard, communication between different elements may be accomplished using an available wired and/or wireless connection and/or according to any suitable communication (and/or network) standard or protocol. Alternatively or additionally, the data may be indirectly loaded into the system 10. For example, the data may be stored in a suitable machine-readable medium (for example, a flash memory card), and then the medium is used to load the data into the system 10 (for example, by a user or an authorized personnel of the system on site), or the data may be downloaded to an electronic device (for example, a laptop) capable of local communication, and then the device is used on site (for example, by a user or an authorized personnel of the system) to upload the data to the system 10 by means of a direct connection (for example, a USB connector).

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

The present application is described above with reference to specific embodiments. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred embodiments of the present application are described above with reference to the accompanying drawings. Many features and advantages of the implementations are clear according to the detailed description, and therefore the appended claims are intended to cover all these features and advantages that fall within the true spirit and scope of these implementations. In addition, as many modifications and changes could be easily conceived of by those skilled in the art, the embodiments of the present application are not limited to the illustrated and described precise structures and operations, but can encompass all appropriate modifications, changes, and equivalents that fall within the scope of the implementations.

1. A rotation detection method, for performing rotation detection on a scanning gantry driven by a motor, characterized by comprising:
detecting at least one rotation of the scanning gantry by using a detector provided on the scanning gantry, and generating a pulse signal;
counting rotations of the motor by using an encoder provided on the motor; and
determining whether a rotation fault has occurred according to the number of counts within a time between two consecutive pulse signals.

2. The rotation detection method according to claim 1, wherein the detector generates a pulse signal of one period each time the scanning gantry performs one complete rotation.

3. The rotation detection method according to claim 1, further including, when it is determined that a rotation fault has occurred in the scanning gantry, stopping rotation of the scanning gantry and aborting a scan.

4. The rotation detection method according to claim 1, wherein the determining whether a rotation fault has occurred includes:
comparing the number of counts within the time between the two consecutive pulse signals with a first threshold; and
when a comparison result is not within a first predetermined range, determining that a rotation fault has occurred.

5. The rotation detection method according to claim 4, further including:
determining a rotation mode of the scanning gantry; and
adjusting, according to the determined rotation mode of the scanning gantry, the first threshold corresponding to the comparison.

6. The rotation detection method according to claim 5, wherein rotation modes of the scanning gantry comprise a constant speed mode, an acceleration mode, and a deceleration mode, and wherein the constant speed mode corresponds to a preset threshold V11, the acceleration mode corresponds to a preset threshold V12, the deceleration mode corresponds to a preset threshold V13, V12=K11*V11, and V13=K12*V11, wherein K11 and K12 are adjustment coefficients, and/or the rotation modes of the scanning gantry comprise a first constant speed mode, a second constant speed mode, and a third constant speed mode; the first constant speed mode corresponds to a preset threshold V21, the second constant speed mode corresponds to a preset threshold V22, the third constant speed mode corresponds to a preset threshold V23, V22=K21*V21, and V23=K22*V21, wherein K21 and K22 are adjustment coefficients.

7. The rotation detection method according to claim 4, further including:
totaling a plurality of comparison results; and
when a totaling result is not within a second predetermined range, determining that a rotation fault has occurred.

8. The rotation detection method according to claim 7, wherein when a rotation mode of the scanning gantry is a constant speed mode, the totaling is enabled, and when the rotation mode of the scanning gantry is not the constant speed mode, the totaling is disabled.

9. A rotation detection apparatus, for performing rotation detection on a scanning gantry driven by a motor, characterized by including:
a pulse generation unit, for detecting at least one rotation of the scanning gantry by using a detector provided on the scanning gantry, and generating a pulse signal;
a motor counting unit, for counting rotations of the motor by using an encoder provided on the motor; and
a fault determination unit, for determining whether a rotation fault has occurred according to the number of counts within a time between two consecutive pulse signals.
